(19) United States
(12) Patent Application Publication
(10) Pub. No.: US 2004/0143334 A1
(43) Pub. Date: Jul. 22, 2004

(54) Artificial disc replacements (ADRS) with features to enhance longevity and prevent extrusion

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/754,042

(22) Filed: Jan. 8, 2004

Related U.S. Application Data
(60) Provisional application No. 60/438,738, filed on Jan. 8, 2003.

(51) Int. Cl. A61F 2/44

(52) U.S. Cl. 623/17.16; 623/17.15

(57) ABSTRACT

Artificial disc replacement (ADR) components allow the use of thicker spacer components, with the goal being to extend the life of the ADR. Various embodiments also tether components within the disc space to prevent extrusion.

### REFERENCE TO RELATED APPLICATION

This application claims priority from U.S. Provisional Patent Application Serial No. 60/438,738, filed Jan. 8, 2003, the entire content of which is incorporated herein by reference.

### FIELD OF THE INVENTION

This invention relates generally to artificial disc replacements (ADRs) and, in particular, to ADRs with features to prevent the extrusion of associated components.

### BACKGROUND OF THE INVENTION

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements to ensure strength and longevity. Such improvements are necessary, since the prosthesis may be subjected to 100,000,000 compression cycles over the life of the implant.

### SUMMARY OF THE INVENTION

This invention improves upon the existing art by providing artificial disc replacement (ADR) components allow the use of thicker spacer components for a given intradiscal spacing, with the goal being to extend the life of the ADR. Various embodiments also tether components within the disc space to prevent extrusion.

An artificial disc replacement (ADR) according to the invention, situated between upper and lower vertebral bodies, includes an endplate affixed to one of the vertebral bodies and a disc spacer situated between the endplate and the other one of the vertebral bodies. In the preferred embodiment, the endplate includes a central region which is thinned or perforated to permit the use of a thicker disc spacer.

The disc spacer may be compressible, though this is not essential to the invention. Other aspects of the invention further include a member coupling the disc spacer to the endplate, to a vertebral body, or to an annulus fibrosis.

### BRIEF DESCRIPTION OF THE DRAWINGS

FIG. 1A is a coronal cross section of an ADR with endplates according to the invention;

FIG. 1B is a top view of an endplate drawn in FIG. 1A;

FIG. 2 is a coronal cross section of an alternative embodiment of the invention with a thin central area;

FIG. 3 is a coronal cross section of the an ADR including a tether device;

FIG. 4 is a sagittal cross section of the spine and an alternative disc spacer tether mechanism;

FIG. 5 is a sagittal cross section of the spine and a further alternative disc space tether device;

FIG. 6A is a sagittal cross section of the spine and a different alternative disc spacer tether device;

FIG. 6B is a sagittal cross section of the spine and the device drawn in FIG. 6A; and

FIG. 7 is an axial cross section of a disc, disc spacer, and alternative devices.

### DETAILED DESCRIPTION OF THE INVENTION

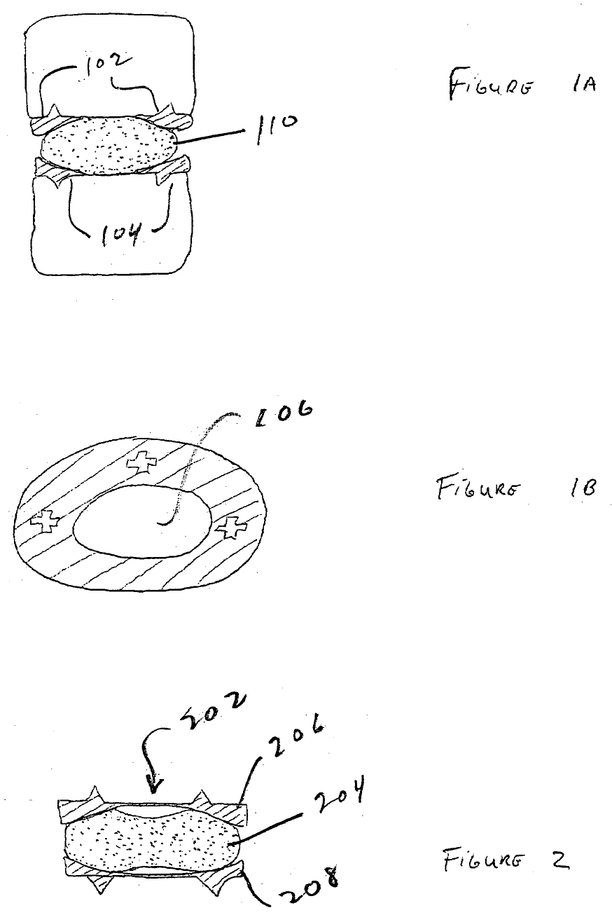

FIG. 1A is a coronal cross section of an ADR according to the invention in conjunction with the spine. In the preferred embodiment, endplates 102, 104 have a central thinned region or void 106 that permits the use of a thicker articulating disc spacer component 110. FIG. 1B is a view of the top of the ADR endplate drawn in FIG. 1A. The articulating disc spacer may be made of polymers such as polyethylene or other suitable material(s), including bags or bladders filled with a gas, liquid, foam or gel, including hydrogels. Although the disc spacer is preferably compressible, it need not be according to the invention.

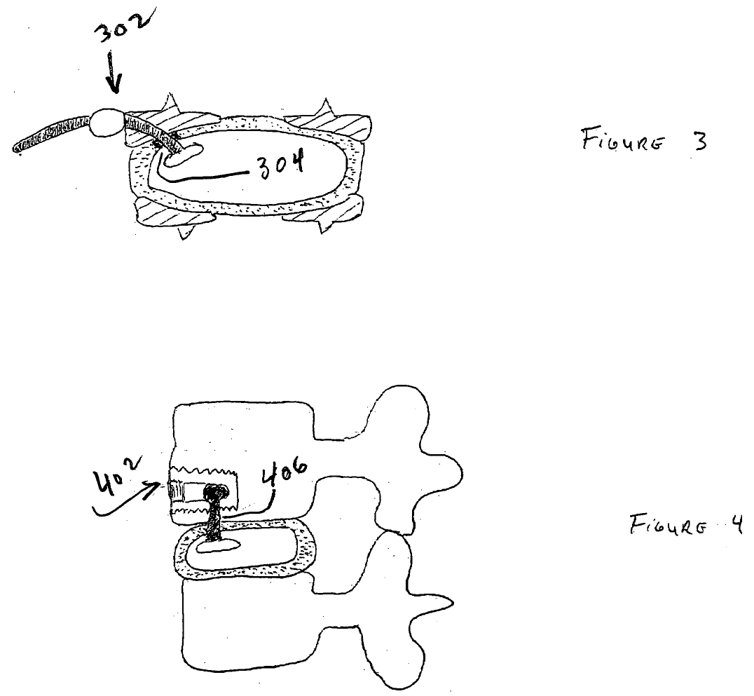

FIG. 2 is a coronal cross section of an alternative embodiment of an ADR having endplates with a thin central area 202. The spacer component 204 may also be shaped as shown to transfer loads from thicker peripheral areas 206, 208 of the ADR endplates. The shape of the spacer component drawn in FIG. 2 may be easier to insert that the taller spacer component drawn in FIG. 1A.

FIG. 3 is a coronal cross section of an ADR according to the invention including a tether device 302 in the form of a cable, string, or other member attached to the disc spacer. In this embodiment the device 302 is threaded through a hole 304 in the ADR endplate. The cable may be cut and crimped after the modular spacer component is inserted between the ADR endplates. The tether mechanism could also be used with ADRs that utilize a single ADR endplate and ADRs that utilize ADR endplates without the central perforation.

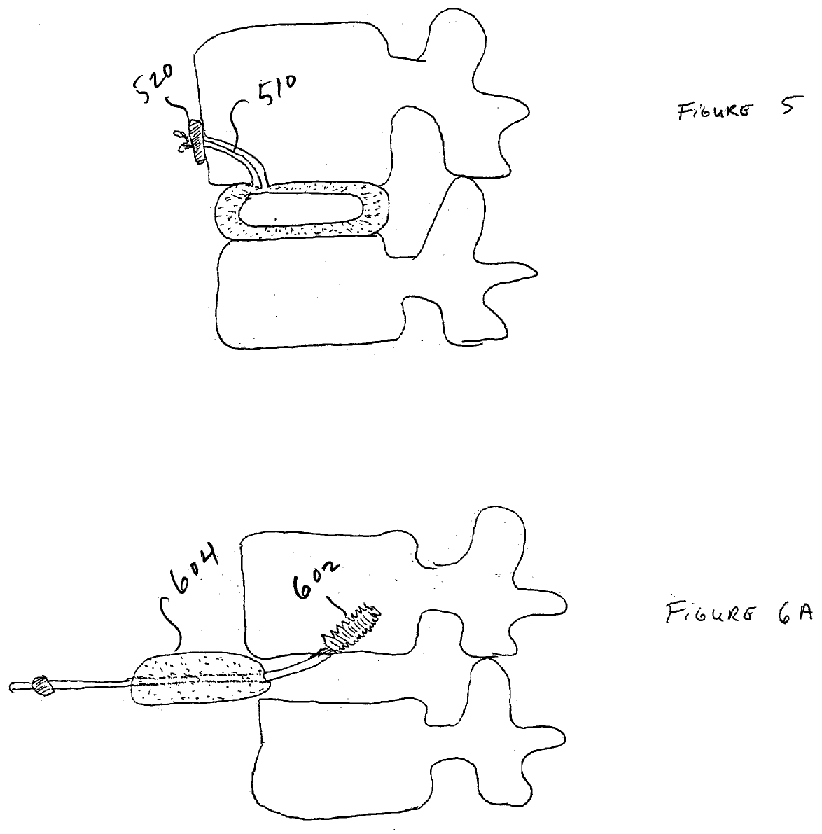

FIG. 4 is a sagittal cross section of the spine and an alternative disc spacer tether mechanism. The cable, string, or other member is preferably placed into a hollow cage-like device 402 that is threaded into a vertebra. A screw is threaded into the cage-like device to contain the disc spacer tether 406.

FIG. 5 is a sagittal cross section of the spine and an alternative disc space tether device. A cable string, or other member(s) 510 attached to the disc spacer, is threaded through a hole fashioned in the vertebra. A button 520 or other device larger that the hole in the vertebra may be used to attach the sutures from the disc spacer to the vertebra.

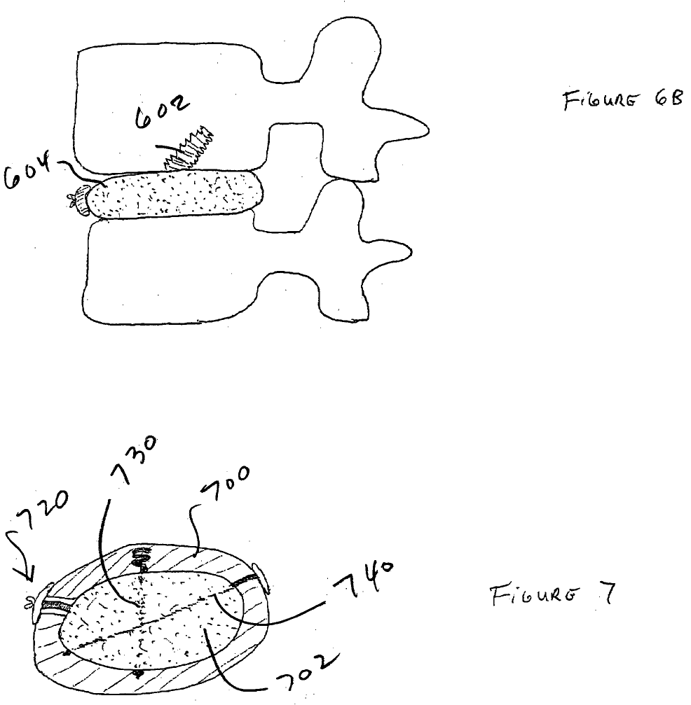

FIG. 6A is a sagittal cross section of the spine and an alternative disc spacer tether device. A cable, suture, or other member attached to a screw 602, is threaded through a disc spacer. The screw is anchored into the vertebra. The disc spacer 604 is slid over the cable and into the disc space. The cable is cut and crimped after the disc spacer is positioned between the vertebrae. FIG. 6B is a sagittal cross section of the spine and the device drawn in FIG. 6A. The tether has been tightened, cut, and fixed to a button or crimp at the side of the spacer.

FIG. 7 is an axial cross section of a disc, disc spacer, and other tether devices according to the invention. The annulus is shown at 700, and the disc spacer, tethered to the annulus, is shown at 702. The device at 720 illustrates the use of a button or other device larger than the hole in the annulus. The tether string or cable from the disc spacer is attached to the device 720.

The device 730 represents a suture or cable that is threaded through the disc spacer and attached to the annulus by a cork screw like device. The cable or suture is tightened, cut and crimped after the disc spacer is placed between the vertebrae.

The device 740 represents a cable, wire, suture or other member that is threaded through the disc spacer and attached to a portion of the member that expands after penetrating the annulus. The cable, wire, or suture courses through the disc spacer. The cable, wire, or suture is tightened, cut, and crimped after the disc spacer is placed between the vertebrae. All of the various embodiments shown, including those depicted in FIG. 7, may be used separately or in combination depending upon the indication.

I claim:

1. An artificial disc replacement (ADR) situated between upper and lower vertebral bodies, comprising:
an endplate affixed to one of the vertebral bodies;
a disc spacer situated between the endplate and the other one of the vertebral bodies; and
wherein the endplate includes a central region which is thinned or perforated to permit the use of a thicker disc spacer.

2. The ADR of claim 1, wherein the disc spacer is compressible.

3. The ADR of claim 1, further including a member coupling the disc spacer to an annulus fibrosis.

4. The ADR of claim 1, further including a member coupling the disc spacer to a vertebral body.

5. The ADR of claim 1, further including a member coupling the disc spacer to the endplate.

6. An artificial disc replacement (ADR) situated between upper and lower vertebral bodies, comprising:
a disc spacer; and
a member coupling the disc spacer to an annulus fibrosis.

7. An artificial disc replacement (ADR) situated between upper and lower vertebral bodies, comprising:
a disc spacer; and
a member coupling the disc spacer to one of the vertebral bodies.

8. An artificial disc replacement (ADR) situated between upper and lower vertebral bodies, comprising:
a disc spacer;
an endplate affixed to one of the vertebral bodies; and
a member coupling the disc spacer to the endplate.
